(19) United States
(12) Patent Application Publication
(10) Pub. No.: US 2022/0074621 A1
(43) Pub. Date: Mar. 10, 2022

(54) TASK AMBIENT HVAC SYSTEM FOR DISTRIBUTED SPACE CONDITIONING

(71) Applicant: Scientific Environmental Design, Inc., High Point, NC (US)

(72) Inventor: Douglas Glass Benefield, Mt. Pleasant, SC (US)

(21) Appl. No.: 17/386,376

(22) Filed: Jul. 27, 2021

Related U.S. Application Data
(63) Continuation of application No. 16/282,131, filed on Feb. 21, 2019, now abandoned.
(60) Provisional application No. 62/671,008, filed on May 14, 2018.

(51) Int. Cl. F24F 11/64; F24F 11/30; H04W 4/021; F24F 11/76; G01N 1/22; G01N 33/00

(52) U.S. Cl. CPC F24F 11/64; F24F 11/30; H04W 4/021; H04L 67/025; G01N 1/2273; G01N 33/0034; G01N 33/0075; F24F 11/76

(57) ABSTRACT

Embodiments of the present disclosure enable an HVAC system comprising an adaptive air distribution system, and methods for distributed and adaptive control of an occupant comfort parameter in a localized, distributed space, within a multizone environment. The system comprises an HVAC system controller, sensors, and actuators for the control and distribution of a primary airflow of an HVAC system into a specific zone of a residential or housing unit. The various system elements are configured to adapt operation under a variable sensible or latent internal load gain or change to achieve a desired occupant comfort parameter target set point or occupant ambient target for a distributed zone within a multizone environment. The occupant comfort parameter target set point is configurable through a task ambient management system residing within an application cloud server operably engaged with an application database, the application cloud server being communicably connected to the HVAC system controller.

### CROSS REFERENCE TO RELATED APPLICATIONS

This application is a continuation of U.S. application Ser. No. 16/282,131 filed Feb. 21, 2019, which claims the benefit of U.S. Provisional Application 62/671,008, filed May 14, 2018; each of which applications are hereby incorporated by reference herein in their entirety.

### FIELD

The present disclosure relates to the field of heating ventilation and air conditioning (HVAC) systems; particularly, a task ambient HVAC system enabling environmental thermal conditioning of distributed occupant spaces within a multizone housing unit.

### BACKGROUND

The level of comfort offered to a commercial or residential occupant is an important aspect of livability. An HVAC system is the primary platform for providing steady-state thermal comfort and acceptable indoor air quality (IAQ) in residential homes and commercial structures. Thermal comfort is that state of mind, which expresses the satisfaction with the thermal environment (ASHRAE). Air distribution is the means of delivering conditioned air to provide comfort in a room. The principal comfort criteria related to the air distribution system are temperature mixing and uniformity. Heating and cooling loads are the measure of energy needed to be added or removed from a space by the HVAC system to provide the desired level of comfort within a space.

With the rising cost of energy, many homeowners appreciate the benefits of owning an energy efficient home. An energy-efficient house is defined as one that is designed and built for decreased energy use and improved occupant comfort through higher levels of insulation, more energy-efficient windows, high efficiency space conditioning and water heating equipment, energy-efficient lighting and appliances, reduced air infiltration, and controlled mechanical ventilation. The addition of energy-efficient windows and insulation to a home have decreased greatly heating and cooling loads. Retrofit measures and new construction practices can achieve lower infiltration levels under increasingly tighter envelopes. Current best practice strives to make homes as airtight as possible using methods such as controlled ventilation, mechanical systems, and energy recovering ventilation (ERV). Air sealing and air-tight construction can provide substantial energy reductions often at the cost of low airflow and ventilation.

The traditional HVAC design aims to create uniform conditions in the entire multizone conditioned space. An energy efficient house with higher levels of insulation, more energy efficient windows, reduced air infiltration, and controlled mechanical ventilation has a lower load, and consequently less air volume is needed to condition the space. The proper amount of air is determined by the room-by-room heating and cooling loads. Too little air delivered to a room results in underheating or undercooling, whereas too much air delivered to a room results in overheating or overcooling. To maintain a uniform temperature and to avoid stratification, the supply air must also adequately mix the air in the room. Mixing of air within the room is a function of the supply outlet and the shape of the air stream entering the room. Throw is the maximum distance the air can reach effectively at a given face velocity and terminal velocity. Air outlets are categorized by the throw as well as the spread (i.e., the width of the air stream).

The air distribution system, air outlet strategy, and a full duct design are critical to the HVAC system delivering the required comfort in an energy efficient house. The amount of air available to provide comfort to the rooms is determined by the equipment selected to meet the loads of a house. Residential air conditioners are often oversized using a design “rule of thumb.” However, oversizing the HVAC system is detrimental to energy use, operational efficiency, cost, comfort, IAQ, residential structure, and equipment wear and durability. Conventional approaches to designing air outlets sized by “rule of thumb” can lead to a level of throw inadequate to provide air mixing in a room to achieve the desired comfort results. The size of the air conditioner affects its cycling behavior and the operation of an oversized unit is characterized by low run-time fraction (RTF) and high cycling rate. Once an HVAC system has been installed, it must be balanced for satisfactory performance. System balancing requires the measurement of actual airflow rates at all supply air outlets and return air inlets and subsequent adjustment of the dampers so that the actual measured flow rate corresponds to the specified flow rates. System balancing may also require adjusting the fan speed to get required temperature drop across the cooling or heating coils and required airflow rates in the conditioned zone. The balancing of an HVAC system that is performed manually can be very expensive and time-consuming. A duct system with unknown pressure drops also makes it difficult to effectively balance the air delivery between rooms since the static pressure drop of each component in the system is unknown.

In energy efficient houses with lower loads, even properly selected equipment will have less capacity to supply air, possibly affecting IAQ, and the performance of the system is dependent upon making the best use of the available centralized air. The risks associated with oversizing the cooling system can be significant because latent loads are typically derived from internal sources and are not substantially reduced by energy-efficiency measures. Internal loads encompass all sensible and latent gains inside a housing envelope, such as occupants, appliances, and electronics. Internal loads can vary widely due to variables such as the number of occupants, and their location and activities. During the cooling season in humid climates, cold and clammy conditions can occur due to reduced dehumidification caused by high cycling. An oversized system that short cycles may not run long enough to sufficiently condense moisture from the air. Excess humidity in the conditioned air delivered to a space may lead to mold growth within the house causing potential health concerns for occupants. Occupants adapt themselves to their thermal environment by changing their clothing insulation, their posture, or their activity. They also adapt their thermal environment to their current requirement by such actions as opening windows, adjusting blinds, and adjusting the heating or cooling set points, affecting the sensible load as well as moisture removal of a total home unit. In general, individual factors such as age, gender, clothing, activity, or body mass of the occupants make it challenging to satisfy the comfort needs of all occupants using a total volume multizone conditioning system.

Through applied effort, ingenuity, and innovation, Applicant has identified deficiencies and problems with existing HVAC systems to optimally control airflow of a residential unit within the constraints of varying internal loads and to provide a desired level of comfort, preferably to a distributed or localized space, within a multizone environment of a housing unit. Applicant has developed a solution that is embodied by the present invention, which is described in detail below.

### SUMMARY

The following presents a simplified summary of some embodiments of the invention to provide a basic understanding of the invention. This summary is not an extensive overview of the invention. It is not intended to identify key/critical elements of the invention or to delineate the scope of the invention. Its sole purpose is to present some embodiments of the invention in a simplified form as a prelude to the more detailed description that is presented later.

An object of the present disclosure is an HVAC system comprising an adaptive air distribution (herein after, “AAD”) system and methods for distributed and adaptive control of an occupant comfort parameter in a localized, distributed space, within a multizone environment. The AAD system comprises one or more HVAC system controller, sensor, and actuator for the control and distribution of a primary airflow of an HVAC system into or out of one or more zone, space, room, or the like, of a housing unit. In various embodiments, the AAD system delivers or diverts one or more primary air source or airflow into one or more secondary air source or airflow. The control and distribution of airflow is determined by a controller and control logic operating in conjunction with one or more sensor, one more actuator within, including but not limited to, one or more HVAC system, sub-system, component, airflow handling unit (AHU), airflow pathway, duct, ductwork, outdoor air inlet-outlet (I-O), space air I-O, distributed space I-O, localized zone I-O, room I-O, or the like. In a preferred embodiment, the AAD system delivers at least one airflow rate into or out of one or more secondary air source or airflow, the secondary airflow adapted to determine an occupant comfort parameter target set point, within a specific, distributed, localized space or zone. In another preferred embodiment, the AAD system delivers at least one airflow rate into or out of one or more secondary air source or airflow, the secondary airflow adapted to determine a thermal ambient target set point within a specific, distributed, localized space or zone. In various embodiments, the occupant comfort parameters include, but are not limited to, one or more temperature, humidity, relative humidity (RH), heating set point, cooling set point, IAQ factor, sensible heat ratio (SHR), luminosity, or the like. In an alternative preferred embodiment, the AAD system delivers a minimum airflow, based on one or more parameters including, but not limited to, an airflow rate, an internal system pressure, outside temperature, indoor temperature, a room temperature, O2 or CO2 concentration, or the like, into one or more secondary air source or airflow, the secondary airflow adapted to determine said occupant comfort parameter or thermal ambient target set point, within a specific, distributed, localized space or zone.

An object of the present disclosure is an AAD system comprising one or more HVAC system controller, sensor, and actuator configured to control at least one desired occupant comfort parameter target set point for at least one zone within a multizone environment. In various embodiments, the HVAC system controller comprises one or more hardware, firmware, or software components, including but not limited to a microcontroller, a microprocessor, a transitory/non-transitory memory storage device (e.g., RAM, ROM, EPROM, etc.), FPGA, I/O module, bus, IC, A/D converter, user interface, display, transceiver (e.g., Bluetooth, Wi-Fi, RF, cellular) module, power source (e.g., battery), IoT device. In a preferred embodiment, the firmware and software (herein “Device App”) components comprise one or more code, source code, object code, microcode, instruction, or logic for data transmission/reception, data processing/analysis, update, communication and feedback control of one or more sensor or actuator. In a preferred embodiment, the controller executes one or more steps or instructions of data transmission/reception, data processing/analysis, update, communication and feedback control of one or more sensor or actuator to control at least one occupant comfort parameter for at least one zone within a multizone environment. In a preferred embodiment, the firmware or software is stored on one or more said transitory/non-transitory memory storage device of the controller. In an alternative embodiment, the Device App is stored on one or more said transitory/non-transitory memory storage device external to the controller. In various embodiments, the AAD system sensors include, but are not limited to, one or more position, velocity, acceleration, rotation, motion (e.g., IR), temperature, humidity, pressure (e.g. total, static, etc.), airflow, current, voltage, acoustic (e.g., piezoelectric), magnetic (e.g., Hall, etc.), electromagnetic (e.g., photodiode, CCD, etc.), luminosity sensor. In a various embodiment, the AAD system actuators include but are not limited to one or more linear, rotational, electric, magnetic, electrical-mechanical, pneumatic, electromechanical relays, or motor. In a preferred embodiment, one or more actuators enable the control of at least one expansion valve, fan, blower, damper, inverter motor, compressor, inverter compressor, flexible duct, or articulating duct actuator. The said sensors, actuators, or components under motion control incorporate at least one transceiver module (e.g., Bluetooth, Wi-Fi, etc.) and at least one local controller, to enable data communication and feedback control.

An object of the present disclosure is an AAD system and methods comprising one or more HVAC system controller, sensor, and actuator configured to adapt operation under variable sensible or latent (e.g., humidity) internal load gain or change to achieve at least one desired occupant thermal comfort for at least one distributed zone within a multizone environment. In various embodiments, the AAD system and its components are configured to adapt operation to control latent and sensible cooling independently to meet a range of internal variable loads. In various embodiments, one or more internal loads are determined using one or more said sensors, IAQ sensor, light sensor, appliance sensor (e.g., refrigerator, dishwasher, small appliance, etc.), sun irradiance sensor, window opening/closing position sensor, water heater sensor, water usage sensor, power usage, power grid sensor, geo-location sensor, outdoor temperature, outdoor humidity, wind speed, wind direction, or occupant sensor. In a preferred embodiment, the one or more occupant sensors include but are not limited to location, motion, activities, metabolic activity, temperature, wearable sensors, personal health/fitness monitor, personal medical diagnostics sensors, personal health devices, mobile computing device, mobile phone, smart phone, sensors of said mobile computing device or phone, or the like. In alternative embodiments, one or more occupant comfort parameter target set point for at least one zone within a multizone environment is determined using one or more said sensors.

An object of the present disclosure is an AAD system comprising one or more HVAC system controller, sensor, and actuator, configured to regulate one or more HVAC system, HVAC component, AHU, and air distribution components in real-time feedback control, according to one or more sensor output, in conjunction with one or more actuators, positioned to distribute or adjust airflow into and or within one or more zones to determine at least one occupant comfort parameter target set point for at least one zone within a multizone environment. In various embodiments, the sensors and actuators enable the modulation of airflow, airflow distribution via one or more outdoor air I-O, inlet, air return inlet, plenum, air I-O, ductwork, ventilation, space I-O, zone I-O, room I-O, into one or more specified, distributed, space or zone, based on an occupant's desired system input. In various embodiments, one or more set point of a specific zone determines the methods and logic to control or modulate one or more fan, blower, dampers to adjust or achieve an air flow rate within said specific zone. In various embodiments, one or more set point of a specific zone determines the logic to control or modulate one or more fan, blower, or dampers to adjust or achieve at least one air flow rate within at least one zone. In various embodiments, one or more set point of a specific zone determines the logic to control or modulate one or more fan, blower, or dampers to adjust or achieve an air flow rate within at least one alternate zone. In various embodiments, the one or more required set point is determined using one or more said sensor output. In one embodiment, one or more zone's airflow and one or more sensor output (e.g., CO2, occupancy) are measured to calculate and deliver a required primary airflow from an outdoor air input. In various embodiments, the control logic calculates a real-time ventilation efficiency from, including but not limited to, occupants, primary airflow, or internal load.

An object of the present disclosure is a task ambient management (herein “TAM”) system comprising an HVAC system controller, an application cloud server operably engaged with an application database, the application cloud server being communicably connected to the HVAC system controller via a wireless network, the controller being operable to communicate the one or more data from at least one of the HVAC system controller, HVAC system, sensor, actuator of said HVAC system to the application cloud server, preferably in real time, via the Internet. In various embodiments, the application cloud server comprises one or more software application (herein “Control App”) operating to send, collect, process, and analyze the one or more HVAC system controller, HVAC system, sensor, and actuators data received and or stored within the application database according to one or more application logic instructions. In various embodiments, a client device (e.g., mobile phone, mobile computing device, PC, etc.) is communicably engaged with the application cloud server, the client device comprising one or more graphical user interface (herein “GUI”) operable to run one or more instance of a TAM system application via a Web browser, the TAM system application configured to deliver or set one or more indoor environmental condition according to the operation of said HVAC controller. In various embodiments, the TAM system application is configured to deliver the status of one or more occupant comfort parameter, including but not limited to, one or more temperature, humidity, relative humidity (RH), heat set point, cooling set point, IAQ factor, sensible heat ratio (SHR), luminosity, minimum airflow rate, or the like. In an alternative embodiment, the TAM system application is configured to deliver or set, under real-time feedback control, one or more indoor environmental condition, thermal ambient target set point, according to one or more user program input, including but not limited to one or more said internal loads, number of occupants, occupant location, occupant demographics (e.g. sex, age, etc.), occupant characteristics (e.g., weight, height, etc.), temperature, humidity, relative humidity (RH), heat set point, cooling set point, IAQ factor, sensible heat ratio (SHR), luminosity, minimum airflow rate, time of day, month, season, address, zip code, geo location, house orientation, longitude, latitude, house characteristics (e.g., number of windows, number of story, number of room, type of room, etc.) or the like. In an alternative embodiment, the TAM system application is configured to deliver, optionally under real-time feedback control, one or more indoor environmental condition, thermal ambient target set point, according to one or more program input, including but not limited to at least one historical or predictive, weather data source, weather forecast, external environment conditioning system, external HVAC system, power grid data, or the like.

Specific embodiments of the present disclosure provide for an adaptive air distribution control system comprising a plurality of indoor air quality sensors, the plurality of indoor air quality sensors being configured to continuously measure one or more indoor air quality parameters; at least one duct actuator being configured to selectively regulate at least one airflow pathway; and, a controller, the controller being operably engaged with the at least one duct actuator and communicably engaged with the plurality of indoor air quality sensors to receive a plurality of indoor air quality data inputs from the plurality of sensors, the controller comprising one or more processors, an input/output interface, and a non-transitory computer readable medium having stored thereon a set of instructions being programmable by a user and executable by the at least one of the one or more processors, to cause the at least one of the one or more processors to perform one or more operations, the set of instructions comprising instructions for configuring one or more HVAC system components in response to the plurality of indoor air quality data inputs from the plurality of sensors; instructions for performing a variable internal load calculation in response to the at least one duct actuator selectively regulating at least one airflow pathway; and, instructions for regulating an electrical current to the one or more HVAC system components in response to the variable internal load calculation.

Further specific embodiments of the present disclosure provide for an adaptive air distribution system comprising an air handler comprising a housing, a variable speed blower, and at least one cooling coil; a plurality of indoor air quality sensors, the plurality of indoor air quality sensors being configured to continuously measure one or more indoor air quality parameters; at least one duct actuator being configured to selectively regulate at least one airflow pathway; and, a controller, the controller being operably engaged with the air handler and the at least one duct actuator, and communicably engaged with the plurality of indoor air quality sensors to receive a plurality of indoor air quality data inputs from the plurality of sensors, the controller comprising one or more processors, an input/output interface, and a non-transitory computer readable medium having stored thereon a set of instructions being programmable by a user and executable by the at least one of the one or more processors, to cause the at least one of the one or more processors to perform one or more operations, the set of instructions comprising instructions for configuring one or more HVAC system components in response to the plurality of indoor air quality data inputs from the plurality of sensors; instructions for performing a variable internal load calculation in response to the at least one duct actuator selectively regulating at least one airflow pathway; and, instructions for regulating an electrical current to the variable speed blower and the at least one cooling coil in response to the variable internal load calculation.

Still further specific embodiments of the present disclosure provide for an adaptive air distribution system comprising an air handler comprising a housing, a variable speed blower, and at least one cooling coil; a plurality of indoor air quality sensors, the plurality of indoor air quality sensors being configured to continuously measure one or more indoor air quality parameters; at least one body-worn or body-carried occupant electronic device being configured to measure one or more occupant comfort parameters; at least one duct actuator being configured to selectively regulate at least one airflow pathway; and, a controller, the controller being operably engaged with the air handler and the at least one duct actuator, the controller being communicably engaged with the plurality of indoor air quality sensors to receive a plurality of indoor air quality data inputs from the plurality of sensors, and being communicably engaged with at least one body-worn or body-carried occupant electronic device to receive a plurality of occupant comfort data inputs, the controller comprising one or more processors, an input/output interface, and a non-transitory computer readable medium having stored thereon a set of instructions being programmable by a user and executable by the at least one of the one or more processors, to cause the at least one of the one or more processors to perform one or more operations, the set of instructions comprising instructions for configuring one or more HVAC system components in response to the plurality of indoor air quality data inputs and the plurality of occupant comfort data inputs; instructions for performing a variable internal load calculation in response to the at least one duct actuator selectively regulating at least one airflow pathway; and, instructions for regulating an electrical current to the variable speed blower and the at least one cooling coil in response to the variable internal load calculation.

The foregoing has outlined rather broadly the more pertinent and important features of the present invention so that the detailed description of the invention that follows may be better understood and so that the present contribution to the art can be more fully appreciated. Additional features of the invention will be described hereinafter which form the subject of the claims of the invention. It should be appreciated by those skilled in the art that the conception and the disclosed specific methods and structures may be readily utilized as a basis for modifying or designing other structures for carrying out the same purposes of the present invention. It should be realized by those skilled in the art that such equivalent structures do not depart from the spirit and scope of the invention as set forth in the appended claims.

### BRIEF DESCRIPTION OF DRAWINGS

The above and other objects, features and advantages of the present disclosure will be more apparent from the following detailed description taken in conjunction with the accompanying drawings, in which:

FIG. 1 is a system diagram of an adaptive air distribution system, according to an embodiment of the present disclosure;

FIG. 2 is a block diagram of the adaptive air distribution system, according to an embodiment of the present disclosure;

FIG. 3 is a process flow diagram for monitoring an internal load and controlling an HVAC system to achieve an occupant comfort parameter target set point, according to an embodiment of the present disclosure;

FIG. 4 is a logic flow diagram for demand zonal control, according to an embodiment of the present disclosure;

FIG. 5 is a task ambient management system diagram, according to an embodiment of the present disclosure;

FIG. 6 is a flow chart of data processing by a Control App of the task ambient management system, according to an embodiment of the present disclosure; and

FIG. 7 is a functional block diagram of a routine for occupant comfort zone environment analysis and control, according to an embodiment of the present disclosure.

### DETAILED DESCRIPTION

Exemplary embodiments are described herein to provide a detailed description of the present disclosure. Variations of these embodiments will be apparent to those of skill in the art. Moreover, certain terminology is used in the following description for convenience only and is not limiting. For example, the words “right,” “left,” “top,” “bottom,” “upper,” “lower,” “inner” and “outer” designate directions in the drawings to which reference is made. The word “a” is defined to mean “at least one.” The terminology includes the words above specifically mentioned, derivatives thereof, and words of similar import.

As used herein the term “task ambient” or “task ambient management system” is defined as any space conditioning system that allows thermal conditions in small, localized zones (e.g., individual room, personal occupant space) to be individually controlled by occupants, while still automatically maintaining acceptable environmental conditions in the central or multizone conditioned space (e.g., space external to an individual room or localized space).

As used herein the term “HVAC” includes systems providing both heating and cooling, heating only, cooling only, as well as systems that provide other occupant comfort and/or conditioning functionality such as humidification, dehumidification, and ventilation.

As used herein the term “residential” when referring to an HVAC system means a type of HVAC system that is suitable to heat, cool and/or otherwise condition the interior of a building that is primarily used as a single-family, a duplex, apartment, office, retail structure or dwelling.

Without loss of generality, some descriptions further herein below will refer to an exemplary scenario in which the innovation is used in a home or housing environment. However, it is to be appreciated that the described embodiments are not so limited and are applicable to use of such innovation in multiple types and locations of HVAC systems.

Embodiments of the present disclosure enable an HVAC system comprising an adaptive air distribution (herein after, “AAD”) system and methods for distributed and adaptive control of an occupant comfort parameter in a localized, distributed space, within a multizone environment. The AAD system comprises a HVAC system controller, sensors, and actuators for the control and distribution of a primary airflow of an HVAC system into one or more zone, space, room, or the like, of a residential or housing unit. The system controller, sensor, and actuator are configured to adapt operation under a variable sensible or latent (e.g., humidity) internal load gain or change to achieve at least one desired occupant comfort parameter target set point or occupant ambient target for at least one distributed zone within a multizone environment. The HVAC system controller, sensor, and actuator are configured to regulate the HVAC system, HVAC component, AHU, and air distribution components, preferably in real-time feedback control, according to sensor measurements and outputs in conjunction with actuators positioned to distribute or adjust airflow, pressure, temperature, into, out of, or within one or more zone to determine at least one occupant comfort parameter target set point or occupant ambient target for at least one zone within a multizone environment. The occupant comfort parameter includes but is not limited to one or more temperature, humidity, relative humidity (RH), heating set point, cooling set point, IAQ factor, sensible heat ratio (SHR), luminosity, or the like. The occupant comfort parameter target set point is adjustable through a task ambient management (herein after “TAM”) system, including but not limited to the HVAC system controller, Device App, client device (e.g., mobile, PC, etc.), a Control App residing within application cloud server operably engaged with an application database, the application cloud server being communicably connected to the HVAC system controller via one or more wireless network including the Internet.

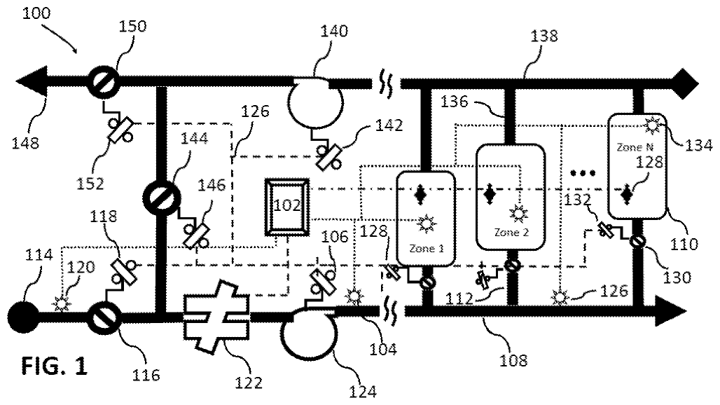

Referring to FIG. 1, a system diagram 100 of an adaptive air distribution system is shown, according to various embodiments. In accordance to an exemplary embodiment, the AAD system of the present disclosure comprises a HVAC system controller 102, one or more sensor, for example sensor 104, and one or more actuator, for example actuator 106, for the control and distribution of a primary supply airflow through primary conduit 108 of an HVAC system into one or more zone, for example zone 110, space, room, or the like, of a housing or residential unit. In one embodiment, sensor 104 may be an O2 sensor or a CO2 sensor. In various embodiments, the HVAC system may include but is not limited to a forced air type heating and cooling system, a radiant heat-based system, heat-pump based systems, and others. In various embodiments, the AAD system delivers one or more primary supply air source or airflow into one or more secondary air source or airflow through one or more secondary conduit, for example duct 112. In various embodiments, the AAD system diverts one or more primary air source or airflow into one or more secondary or fractional air source or airflow starting from an outdoor air flow source 114. The outdoor flow rate may be regulated by a damper 116 under the operational direction of controller 102 through an actuator 118 in conjunction with, for example, airflow sensor 120. In various embodiments, controller 102 are operably engaged (shown as various dotted lines) with one or more actuator, for example actuators 106, 118, with one or more sensors, for example sensor 104, 120, via one or more communication means, including but not limited to direct physical electrical wire connection or wireless connection, employing one or more hardware, analog/digital circuits, ASIC, FPGA, and one or more standard communication protocols. In one embodiment, the regulated outdoor air flow enters and is conditioned by an air handling unit (AHU) 122. In various embodiments, AHU 122 conditions the entering air using one or more components of an HVAC system, including but not limited to heat pump, evaporative coil, cooling coil, dehumidification coil, bypass, heating coil damper, filter, purifier, plenum, cleanser, fan, variable inverter, or the like. The conditioned air flow is then distributed to one or more zone 110 by an air supply fan 124 under the operational direction of controller 102 through an actuator 106. In a preferred embodiment, actuator 106 comprises one or more local controller, further comprising but not limited to a variable frequency driver, variable inverter, a motor, or the like for operation. In an embodiment, the AAD system delivers at least one airflow rate, a secondary airflow, into one or more zone, for example zone 110, based on the output of one or more sensor, for example pressure sensor 126, positioned within primary conduit 108. In a preferred embodiment, the AAD system delivers at least one airflow rate, a secondary airflow, into one or more zone, for example zone 110, based on the input of, for example, a local controller 128, positioned within one or more location of said zone, operating in conjunction with one or more damper, for example damper 130, under the operational direction of controller 102 through an actuator, for example actuator 132. In another preferred embodiment, the AAD system delivers at least one airflow rate, a secondary airflow, into one or more zone, for example zone 110, based on the output of one or more sensor, for example IAQ sensor 134, positioned within one or more location of said zone, operating in conjunction with one or more damper, for example damper 130, under the operational direction of controller 102 through an actuator, for example actuator 132. The one or more secondary airflow rate preferably determines an occupant comfort parameter target set point, within the specific, distributed, localized zone, said parameter set by an occupant using, for example controller 128, said device optionally including a plurality of sensors, the plurality of sensors including, for example, sensor 134 (hereinafter “ambient sensor suite”). Therefore, controller 128 and sensor 134 may be integrated into one unit. In various embodiments, controller 128 may be a thermostat, a smart thermostat, or a mobile computing device. In various embodiments, the secondary airflow exits one or more zone, for example zone 110, via one or more secondary conduit, for example duct 136, and enters a return air conduit 138. One or more fraction of the exiting air flow is then directed and distributed back to AHU 122 or to the outside by an air return fan 140 under the operational direction of controller 102 through an actuator 142. In various embodiments, the exiting airflow distributed back to AHU 120 is regulated by a damper 144 under the operational direction of controller 102 through an actuator 146. In various embodiments, one or more fraction of the exiting airflow distributed to the outside via exhaust air outlet 148 is regulated by a damper 150 under the operational direction of controller 102 through an actuator 152. In various alternative embodiments, the one or more secondary airflow rate is varied, based on the determination or calculation of an internal load and one or more zone load, by one or more said AAD system, HVAC component, AHU, fan, blower, one or more HVAC controller, sensor, actuator, conduit, duct, and combinations thereof.

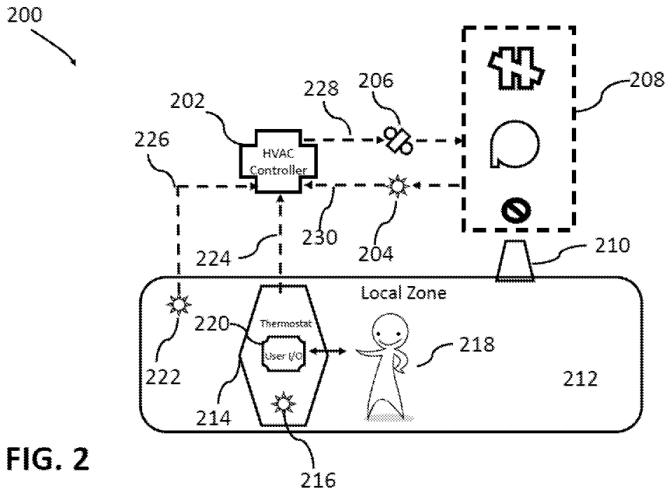

FIG. 2. shows a block diagram of AAD system 200, according to various exemplary embodiments. The AAD system comprises one or more HVAC system controller 202, corresponding to 102 of FIG. 1, feedback sensor 204, actuator 206 configured to actuate and control a suite 208 of components including at least one AHU, blower, fan, and damper, preferably one damper located within a duct 210 in proximity to an air outlet for conditioning a local zone 212. The AAD system also comprises a local controller, such as a thermostat 214, the local controller comprising at least one environment sensor, for example temperature or humidity sensor 216. One or more environment comfort set point of local zone 212 may be determined by an occupant 218 via an input-output (I/O) interface 220 of controller 214. In various embodiments, one or more ambient sensor suite 222 may operate independently from controller 214 to provide feedback to AAD controller 202. Ambient sensor suite 222 may comprise one or more sensor, including but not limited to occupant, temperature, pressure, airflow, humidity, gas, IAQ, particulate, biologic, microbe, mold, bacteria, radiation, luminosity, or the like. In an alternative embodiment, ambient sensor suite 222 may include a(n): appliance sensor (e.g., refrigerator, dishwasher, small appliance, etc.); sun irradiance sensor; window open/close position sensor; water heater sensor; water usage sensor, power usage; or the like. Typically, thermostat 214 is mounted on a wall within zone 212. Thermostat 214 communicates with controller 202 to send one or more user input, for example a user-defined comfort set point, one or more sensor measurement from sensor 216, or combinations thereof. The controller 202 uses one or more input signal 224, 226 to generate one or more control signal 228 causing one or more actuator 206 to operate one or more components or unit of suite 208, individually, in combination, in concert, sequentially, or simultaneously to control at least one desired occupant comfort parameter target set point for at least one zone, for example zone 212, within a multizone environment. The at least one parameter target set point may be reached by controller 202 via one or more feedback signal 230 received from one or more sensor 204.

In various embodiments, the HVAC system controller 202 comprises one or more hardware, firmware, software components, including but not limited to a microcontroller, a microprocessor, a central processing unit (CPU), FPGA, a transitory/non-transitory memory storage device (e.g., RAM, ROM, EPROM, etc.), I/O module, bus, interface bus, IC, A/D converter, user interface, display, transceiver (e.g., Bluetooth, Wi-Fi, RF, cellular) module, network interface, power source (e.g., battery). The power source may be of any standard form, AC or DC power (e.g., solar photovoltaic), for powering small electronic circuit board devices, including but not limited to: alkaline, lithium hydride, lithium ion, lithium polymer, nickel cadmium, solar cells, the like, or combinations thereof. It is understood that any suitable power source may be used without departing from the scope of the present teachings.

In various embodiments, the microcontroller, microprocessor or CPU comprises at least one high-speed data processor. The said processor may incorporate one or more but not limited to integrated system (bus) controllers, memory management control units, floating point units, digital signal processing (DSP) units, and or the like. In various embodiments, the microcontroller may be, but is not limited to, an Intel MCS 51 (i.e., 8051 microcontroller). In various embodiments, the CPU may be a microprocessor, but is not limited to a (n): AMID Athlon, Duron, Opteron; ARM's application; IBM and or Motorola Dragon Ball and PowerPC; IBM and Sony Cell processor; Intel Celeron, Core (2) Duo, Itanium, Pentium, Xeon, XScale processor or the like; or combinations thereof. In various embodiments, various features and functions of controller 202 may be implemented via one or more said microprocessor and or via embedded components (e.g., ASIC, DSP, FPGA, etc.). The embedded components may comprise, but are not limited to, hardware, middleware, software, and or combinations thereof. For example, the controller capabilities discussed herein may be achieved with FPGAs, semiconductor devices containing programmable logic components. It is understood that any suitable processor may be used without departing from the scope of the present teachings.

The HVAC controller 202 and or a computing system of the present disclosure may employ various forms of memory storage device. In various configurations, memory storage may comprise one or more transitory/non-transitory memory storage device, including but not limited to read-only memory (ROM), random-access memory (RAM), static RAM, dynamic RAM, erasable programmable ROM (EPROMP), EEPROM, flash, any conventional computer system storage, a drum, a (fixed and/or removable) magnetic disk drive, a magneto-optical drive, optical drive (e.g., Blueray, CD ROM/RAM/Recordable (R)/Re-Writable (RW), DVD R/RW, HD DVD R/RW etc.), an array of devices (e.g., Redundant Array of Independent Disks (RAID)), solid state memory devices (e.g., USB, solid state drives (SSD), etc.), other processor-readable storage means, and the like, or combinations thereof. It is understood that any suitable memory storage device may be used without departing from the scope of the present teachings.

In various embodiments, controller 202 may incorporate one or more network interfaces, to accept, communicate, and or connect to one or more communication network, providing operation access or control of a sensor, local controller, actuator, HVAC component, HVAC unit, HVAC system, remote server, cloud server, remote database, cloud database, remote client, or the like. The network interfaces may employ connection protocols or standards including, but not limited to: direct connect, Ethernet (thick, thin, twisted pair 10/100/1000/10000 Base T, and/or the like), Token Ring, wireless connection such as IEEE 802.11a-x, and the like, or combinations thereof. A distributed network controllers or architectures may similarly be employed to pool, load balance, and/or otherwise decrease/increase the communicative bandwidth as necessary. A communication network may be any one and/or the combination of the following: a direct interconnection; a Local Area Network (LAN); a wide area network (WAN); a Metropolitan Area Network (MAN); public network; the Internet; a wireless network (e.g., Bluetooth, Wi-Fi, cellular, 2G, 3G, 4G, 5G, LITE, etc.); and or the like. It is understood that any suitable network interface, network, network communication protocol, and network communication standards may be used without departing from the scope of the present teachings.

In various embodiments, the HVAC controller 202 and local controller 210 may comprise alternative Internet of Things (IoTs), hardware, firmware, software, physical network, network protocols, data protocols, architecture, framework, standards, applications, and APIs to sense, diagnose, analyze, and transmit data to a remote application server and database. These alternative implementations include but are not limited to TSN Ethernet (802.1, 802.3) Wireless PAN (802.15, 802.15.4), Wireless LAN (802.11 Wi-Fi), Wireless 2G/3G/4G/5G/LTE, Wireless WLAN (802.16), Thread, Zigbee, WirelessHART, Bluetooth, BLE, Wi-Fi, LTE, Internet Protocol (IP), IPv6, IPv6 over Low-Power Wireless Area Network (6LoWPAN), IP6 over BLE, TCP, UDP, TCP, DDSI-RTPS, CoAP, MQTT, NFC, HTTP, DDS, TLS, DTLS, oneM2M, Web Services, GATT protocol, ATT protocol, Representational State Transfer (REST) APIs, Lightweight M2M, SOAP, HL7, HL7 CDA, IEEE 11073 DIM, or the like. Exemplary platforms include but are not limited to TI CC2538, nFR52832, TI MSP430x, Atmel AVR, Freescale MC1322x, Arduino, Quark D200, CC2650, NXP FRDM, Hexiware, nRF52, ST Nucleo, Imote2, Shimmer, IRIS, Telos Rev B, MicaZ, Mica2, Mica2dot, Mulle, TinyNode, Zolertia Z1, UCMote Mini, nRF52840, nRF51 DK, BMD-300-EVAL-ES, STM32F4DISCOVERY, STM32-E407, Arduino Zero, Arduino Zero Pro, NUCLEO-F401RE, PIC32MX470, PIC32MZ2048EFG100, Arduino-due, UDOO, CC2538DK, OpenMote, pca10005, yunjia-nrf51822, STM32 Nucleo32, telosb, chronos, Altera, Atmel, Cortus, Freescale, Infineon, Microsemi, NXP, Renesas, TI, ST, Intel, Xilinx, Nordic nRF52DK, Seed Arch Link, Realtek RT8195AM, Wizwiki, EFM32, NUCLEO F334R8, hexiwear, mbuino, mbedLPC.

In various embodiments, HVAC system controller 202 may be programmed with one or more firmware and or software (herein “Device App”) components. The firmware or software comprises, but is not limited to, one or more code, source code, object code, microcode, instruction, or logic for data transmission/reception, data processing/analysis, update, communication and feedback control of at least one unit of suite 208 via one or more sensor 204 or actuator 206. The controller 202 may store in a said memory storage unit one or more operating system component, an executable program facilitating the operation of the controller. The typical operations may include but are not limited to access of I/O, network interfaces, peripheral devices, storage devices, power devices, and the like, or combinations thereof. The operating system may be a highly fault tolerant, scalable, and secure system, including but not limited to: Apple Macintosh OS X (Server); Apple Macintosh OS; AT&T Plan 9; Be OS; IBM OS/2, Microsoft DOS, Microsoft Windows 2000/2003/3.1/95/98/CE/Millennium/NT/Vista/XP (Server), Palm OS; Unix and Unix-like system distributions (e.g., AT&T's UNIX; Berkley Software Distribution (BSD) variations such as FreeBSD, NetBSD, OpenBSD; Linux distributions such as Red Hat, Ubuntu); the like operating systems; or combinations thereof. The operating system may provide communications protocols that allow the controller to communicate with other entities through a communications network. Various communication protocols may be used by said controller as a subcarrier transport mechanism for interaction, including, but not limited to: multicast, TCP/IP, UDP, unicast, the like, or combinations thereof. In a preferred embodiment, the controller 202 executes one or more steps of data transmission/reception, data processing/analysis, update, communication and feedback control of one or more sensor 204, 216, ambient sensor suite 222 or actuator 206 to control at least one occupant 218 comfort parameter for at least one zone 212 within a multizone environment. In a preferred embodiment, the firmware or software is stored on one or more of said transitory/non-transitory memory storage device of the controller 202. In an alternative embodiment, the Device App is stored on one or more of said transitory/non-transitory memory storage device external to the controller, for example on a cloud server. In various embodiments, the AAD system sensors 204, 216, and ambient sensor suite 222 include, but are not limited to, one or more position, velocity, acceleration, rotation, motion (e.g., IR), temperature, humidity, pressure (e.g. total, static, etc.), airflow, pitot tube, current, voltage, acoustic (e.g., piezoelectric), magnetic (e.g., Hall, etc.), electromagnetic (e.g., photodiode, CCD, etc.), luminosity, gauge, and sensor. In a various embodiment, the AAD system actuators 206 include but are not limited to one or more linear, rotational, electric, magnetic, electrical-mechanical, pneumatic, electromechanical relays, and motor. In a preferred embodiment, one or more actuators 206 enable the control of at least one component of suite 208, at least one expansion valve, fan, blower, damper, inverter motor, inverter driver, compressor, inverter driven compressor, variable frequency driven fan, variable frequency driven fan, flexible duct, and articulating duct actuator of an HVAC system. The said sensors, actuators, or components under motion control may incorporate at least one transceiver module (e.g., Bluetooth, Wi-Fi, etc.), IoT device, at least one network communication interface, at least one local controller, to enable data communication and feedback control. The said Device App controls a variety of tasks of the AAD system via controller 202. The Device App may collect data from sensors, for example, ambient sensor suite 222 and feedback sensor 204 and transmit them to a Control App over the network residing on a remote cloud server. The Device App may also execute commands received from the Control App.

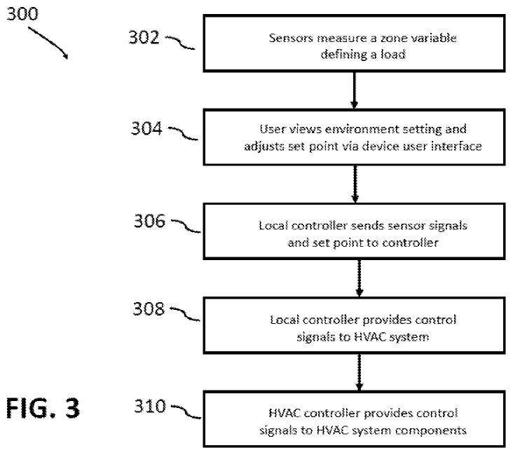

Referring now to FIG. 3, a process 300 for monitoring an internal load and controlling an HVAC system to achieve at least one occupant comfort parameter target set point is shown, according to an exemplary embodiment. Process 300 may be performed by the Device App of AAD system 200, as described herein with reference to FIG. 2. In process 300, local controller 214 or one or more ambient sensor suite 222 provides one or more measurements of a variable within zone 212 (step 302). A variable may be a temperature or humidity level within zone 212. User 218 views the measured temperature and adjusts the temperature set point via user interface 220 of controller 214 (step 304). The controller 214, under instructions of the Device App, sends the measured temperature and the set point to controller 202 (step 306). Controller 214 uses the measured temperature and the set point to generate and provide one or more control signals to one or more components of suite 208 comprising an HVAC system (step 308). The HVAC system of the present disclosure operates in accordance with one or more control signal 228 and one or more feedback signal 230 to provide a required heating/cooling load to zone 212. In various embodiments, the HVAC system controller, sensor, and actuator are configured to adapt operation under variable sensible or latent (e.g., humidity) internal load gain or change to achieve at least one desired occupant for at least one distributed zone 212 within a multizone environment. In various embodiments, the AAD system and its components are configured to adapt operation to control latent and sensible cooling independently to meet a range of internal variable loads, sensed by one or more sensor 204, 216, and ambient sensor suite 222. In various embodiments, one or more internal loads are determined using one or more said sensors, IAQ sensor, light sensor, appliance sensor (e.g., refrigerator, dishwasher, small appliance, etc.), sun irradiance sensor, window open/close position sensor, water heater sensor, water usage sensor, power usage, power grid sensor, geo-location sensor, outdoor temperature, outdoor humidity, wind speed, wind direction, and occupant sensor. In a preferred embodiment, the one or more occupant sensors include but are not limited to location, motion, activities, metabolic activity, temperature, wearable sensors, personal health/fitness monitor, personal medical diagnostics sensors, personal health devices, mobile computing device, mobile phone, smart phone, sensors of said mobile computing device or phone, or the like. In alternative embodiments, one or more occupant comfort parameter target set point for at least one zone, for example zone 212, within a multizone environment is determined using one or more said sensors 204, 216, and ambient sensor suite 222.

An object of the present disclosure is an AAD system, for example AAD system 200 comprising one or more HVAC system controller 202, actuator 206, and sensor 230, configured to regulate one or more HVAC system, HVAC component, AHU, and air distribution components suite 208 in real-time feedback control, according to one or more sensor output, in conjunction with one or more actuators, positioned to distribute or adjust airflow into and or within one or more zone, for example zone 212, to determine at least one occupant 218 comfort parameter target set point for at least one zone within a multizone environment. One or more control methods may be employed to achieve the occupant parameter target set point. In one embodiment, the AHU may be controlled to provide a constant static pressure set point, within one or more primary or secondary airflow pathway, one or more duct, vent, air inlet, air out, or the like. In various embodiments, the said static pressure may be changed or reset based on, but not limited to, an outside air (OA) temperature, humidity, pressure, airflow rate. In an alternative embodiment, the said static pressure may be changed or set based on modulation of one or more HVAC components, including but not limited to cooling/heating coil, evaporative coil, dehumidifying coil, variable fan, blower, or damper. In various embodiments, the AO airflow set point may be changed, modified, or reset to maintain an acceptable space or zone IAQ, oxygen (O2) level, or a carbon dioxide (CO2) level. In one embodiment, one or more zone's airflow and one or more sensor output (e.g., CO2, occupancy) are measured to calculate and deliver a required primary airflow from an OA input. In one embodiment, one or more zone's airflow and one or more sensor output (e.g., CO2, occupancy) are measured to calculate and deliver a required ventilation airflow. In various embodiments, the sensors and actuators enable the modulation of airflow, airflow distribution via one or more outdoor air I-O, inlet, air return inlet, plenum, air I-O, ductwork, ventilation, space I-O, zone I-O, room I-O, into one or more specified, distributed, space or zone, based on an occupant's desired system input. In various embodiments, one or more set point of a specific zone determines the methods and logic to control or modulate one or more fan, blower, and dampers to adjust or achieve an air flow rate within said specific zone. In various embodiments, one or more set point of a specific zone determines the logic to control or modulate one or more fan, blower, and dampers to adjust or achieve at least one air flow rate within at least one zone. In various embodiments, one or more set point of a specific zone, determines the logic to control or modulate one or more fan, blower, and dampers to adjust or achieve an air flow rate within at least one alternate zone. In various embodiments, the one or more required set point is determined using one or more said sensor output. In various embodiments, the control logic calculates a real-time ventilation efficiency from, including but not limited to, occupants, primary airflow, or internal load. In various embodiments, the control logic employed is based on one or more logic flow described herein before, after or throughout the present disclosure.

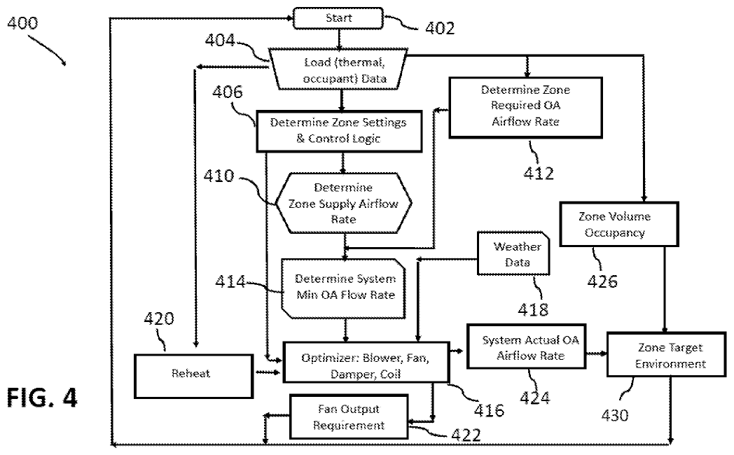

FIG. 4 shows a logic flow diagram 400 illustrating an exemplary embodiment for demand zonal control. A method is provided with an initial start 402 step for an energy efficient means to achieve a zone environment comfort set point target, preferably by controlling one or more actuators, preferably a blower fan, more preferably a local damper, for example damper 130 of FIG. 1, most preferably combinations thereof. By way of example only and not by way of limitation, the zone environment comfort target may be a minimum acceptable or optimum indoor air quality (IAQ). First, one or more load (e.g., thermal, occupancy) is determined via sensors or calculated for a specific zone (step 404). Second, the zone settings are derived from a local sensor or controller and control logic is determined that is appropriate for a user chosen comfort set point (step 406). In various embodiments, the control logic incorporates one or more discharge airflow rate determined based on one or more zonal temperature, set point temperature, and/or temperature integral or differential calculus. In various alternative embodiments, the control logic incorporates one or more discharge airflow rate determined based on one or more system or local static pressure, static pressure set point, and/or static pressure integral or differential calculus. In various alternative embodiments, the control logic incorporates one or more discharge airflow rate determined based on one or more system airflow rate or local airflow rate, airflow rate set point, and or airflow rate integral or differential calculus. In various alternative embodiments, the control logic incorporates one or more discharge airflow rate determined based on one or more zonal humidity, humidity level (HR %) set point, and or humidity integral or differential calculus. Third, the zone supply air flowrate is determined based on the zone settings (step 410). Concurrently, the zone required outdoor air (OA) flowrate is also determined based one or more internal or external load data acquired during step 404. In step 414, a system minimum airflow rate is determined based on the input of step 414 and step 412. In various embodiments, the zone required OA airflow rate of step 412 is determined based on one or more sensed or calculated occupancy schedule (e.g., time of day, week, month, etc.), area, or zone occupancy density. In various embodiments, the minimum OA flow rate of step 414 is determined based on one or more of, but not limited to, sensed or calculated, integral or differential calculus, zone outdoor air ratio, input from step 410, and input from step 412. Fourth, an optimizer model may be implemented (step 416) whereby data for an external variable or load (e.g., weather) is collected (step 418) and a reheat option (step 420) to reach a target temperature set point, as additional inputs. In addition, said optimizer model determines a blower/fan, damper, or combinations thereof output requirement for the HVAC system (step 422). In various embodiments, the optimizer model of step 416 comprises one or more control logic to minimize the energy consumption of one or more HVAC components (e.g., blower, coil, fan, etc.) while provisioning an optimal OA airflow rate, a minimum airflow rate, into one or more local zone of a multizone environment. In various embodiments, the optimizer provisions an optimal OA airflow rate based on one or more, but not limited to, input from step 406, step 414, step 418, and step 420. Fifth, a system actual OA flowrate or airflow is delivered into a zone (step 424). Finally, a zone and system IAQ level set point is reached (step 430) based one or more input steps, including but not limited to a zone volume occupancy sensed or calculated step (step 426), one or more IAQ model, optionally an CO2 model, determined using one or more non-limiting integral or differential calculus. In a preferred embodiment, a CO2 based dynamic control logic is employed that calculates the required set point for the system OA rate and then modulates one or more dampers of an AHU, for example AHU 122 of FIG. 1, or one or more local dampers, for example damper 130 of FIG. 1, within one or more local duct, ductwork, to maintain the OA flowrate, inside airflow, at a new set point. The process continues iteratively starting at step 402 until a zone environment comfort set point target is reached or achieved as desired by an occupant. It is understood that the process can be applied or performed by one or more Device App and Control App of the present disclosure to achieve any said zone environment comfort set point target, or ambient target set point, without departing from the scope of the present teachings.

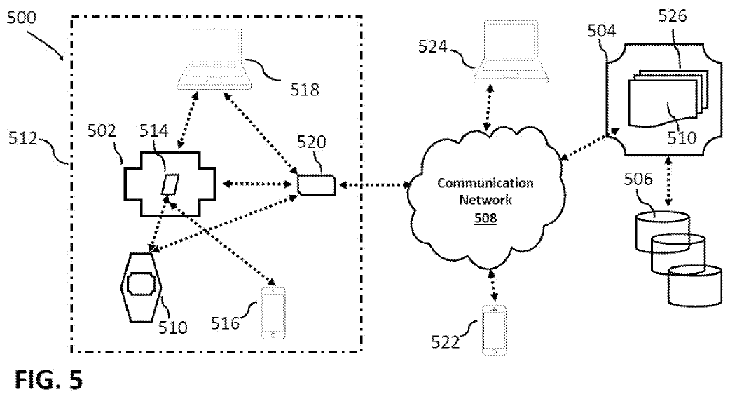

Referring to FIG. 5, a task ambient management (herein “TAM”) system diagram 500 is shown, according to various exemplary embodiments of the present disclosure. TAM system 500 comprises an HVAC system controller 502, an application cloud server 504 operably engaged with an application database 506, the application cloud server being communicably connected (shown as dotted arrows) to the HVAC system controller 502 via a communication network 508, the controller being operable to communicate the one or more data from at least one HVAC system controller, local controller 510, HVAC system and components located within housing or residential environment 512, to the application cloud server 504, preferably in real time, via network 508, for example the Internet. In various embodiments, controller 502 comprises a network communication module 514 to enable communication (shown as dotted arrows) of data, optionally via a LAN, to/from one or more external communication device including a mobile computing 516, a personal computing device (PC) 518, or a gateway controller/router computing unit 520 (facilitating connection to external network 508, and ultimately to server 504), said units preferably located at one or more locations within residential or housing environment 512. In various embodiments, an occupant of environment 512 can directly program and control, from one or more zone 212, the operation of HVAC controller 502 or local controller 510 via said LAN, optionally via router 520, using a mobile computing device 516 or PC 518. In various embodiments, the application cloud server 504 comprises one or more software application (herein “Control App”) 510 operating to send, collect, process, and analyze the one or more HVAC system controller 502, HVAC system, sensor, actuators data received and to store within one or more application database 506 according to one or more application logic and or instructions. In various embodiments, the Control App monitors one or more remote procedural call (RPC) connections from the said Device App. Each call from the Device App updates one or more data point, including but not limited to, sensor input/out, actuator input/output, or indoor environmental conditions, task ambient conditions, optionally average, standard deviation, standard error values. In various embodiments, the Control App then stores these values to one or more database 506 for further processing or analysis. In various embodiments, the Control App sends one or more of, but not limited to, control signal, control logic, control law, control instructions, based on at least one sensor data, and enables invocation of one or more appropriate operation, analytical, calibration, or diagnostic procedures on the Device App. In various embodiments, one or more client device (e.g., mobile phone 522, PC 524, etc.) is communicably engaged with the application cloud server 504, the client device comprising one or more graphical user interface (herein “GUI”), preferably implemented via one or more Web browser application, interface, or the like, being operable to enable user registration, authentication, configuration of features, operations, operation schedule, calibration, diagnostic, maintenance schedule, of one or more HVAC controller, local controller, HVAC system, HVAC system components, sensors, ambient sensor suite, and actuators. In various embodiments, one or more GUI enables a user to run one or more instance of a TAM system application 526 via a Web App, the TAM system application 526 configured to deliver or set one or more indoor environmental condition according to the operation of said HVAC controller 502. In various embodiments, the TAM system application 526 is configured to deliver the status of one or more occupant comfort parameters, including but not limited to one or more of temperature, humidity, relative humidity (RH), heat set point, cooling set point, IAQ factor, sensible heat ratio (SHR), luminosity level, minimum airflow rate, or the like. In an alternative embodiment, the TAM system application 526 (e.g., one or more alternative Control App) is configured to deliver or set, under real-time feedback control, one or more indoor environmental condition, and thermal ambient target set point, according to one or more user program input, including but not limited to one or more of said internal loads, number of occupants, occupant location, local zone, multizone, occupant schedule, occupant zone density, occupant demographics (e.g. sex, age, etc.), occupant characteristics (e.g., weight, height, etc.), temperature, humidity, relative humidity (RH), heat set point, cooling set point, IAQ factor, sensible heat ratio (SHR), luminosity, minimum airflow rate, time of day, month, season, address, zip code, geo location, house orientation, longitude, latitude, house characteristics (e.g., number of windows, number of story, number of room, type of room, etc.) or the like. In an alternative embodiment, one or more TAM system application 526 is configured to deliver, optionally under real-time feedback control, one or more indoor environmental condition, thermal ambient target set point, according to one or more program input including but not limited to, at least one of historical or predictive weather data source, weather forecast, external environment conditioning system, external HVAC system, power grid data, or the like. In various embodiments, the TAM system application 526 is configured to deliver the status of one or more occupant comfort parameter, HVAC system configuration, operation, diagnostics, fault signal or the like, housing unit status, status of an appliance, internal-external load condition, weather, weather prediction, to one or more client device via one or more, including but not limited to, message, SMS text message, an image, a graphical output, a video, the like, or combinations thereof.

In various embodiments, the one or more said client device enables access to or is configured to execute on one or more of a web browser (e.g., Internet Explorer, Firefox, Chrome, Safari) or other rendering engine that, typically, is compatible with AJAX technologies (e.g., XHTML, XML, CSS, DOM, JSON, and the like). AJAX technologies include XHTML (Extensible HTML) and CSS (Cascading Style Sheets) for marking up and styling information, the use of DOM (Document Object Model) accessed with client-side scripting languages, the use of an XMLHttpRequest object (an API used by a scripting language) to transfer XML and other text data asynchronously to and from said server using HTTP), and use of XML or JSON (Javascript Object Notation) as a format to transfer data between the server and the client. In a web environment, an end user accesses the site in the usual manner, i.e., by opening the browser to a URL associated with a service provider domain. The user may authenticate to the site by entry of a username and password. The connection between the end user entity machine and the system may be private (e.g., via SSL). The server side of the system may comprise conventional hosting components, such as IP switches, web servers, said application servers, administration servers, databases, or the like. Where AJAX is used on the client side, the client-side code (an AJAX shim) may execute natively in the web browser of the end user or other rendering engine. Typically, this code is served to the client machine when the end user accesses the site, although in the alternative it may be resident on the client machine persistently. Finally, it is understood that any web browser, rendering engine, present or future version, may be used in conjunction with one or more said HVAC system, controller, connected device, sensor, sensor suite, application server, or database without departing from the scope of the present teachings.

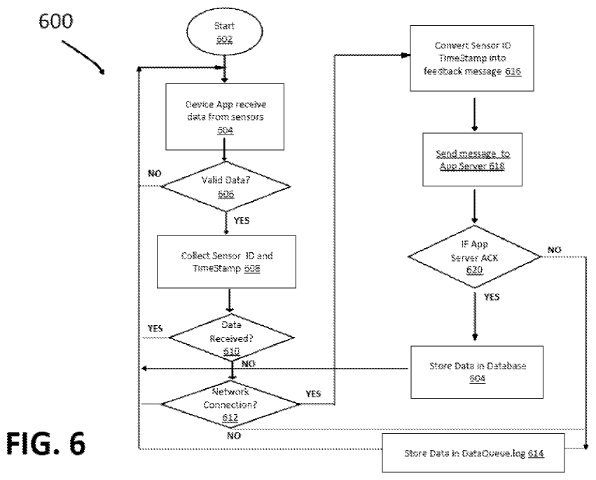

Referring now to FIG. 6, a flow chart 600 of data processing by a Control App 510 of the TAM system 500 is shown. At the Start 602, the Device App receives data from at least one local thermostat 214 or ambient sensor suite 222 of FIG. 2 (step 604). The validity of local controller 214 or ambient sensor suite 222 data is then determined, with a NO decision leading to the initiation of another query from Start 602 (step 606). If a YES determination is made (step 606), then Device App proceeds to collect a Sensor ID and TimeStamp (step 608) from one or more local controller 214 or sensor suite 222. Another decision is made as to whether the collected data has been received before (step 610). If NO, then Device App proceeds to determine whether one or more communication network 508 is available (step 612). If NO, then Device App proceeds to Store Data (step 614) where the data is then stored in a queue file for later transmission to application server 504. If YES, then Device App proceeds to convert the data into a feedback message (step 616) and then sends the message to application server 504 (step 618) to be further processed by Control App 510. A decision is made to confirm that application server 504 has received data (step 620). If a NO acknowledgement is received, then Device App stores the data for later transmission (step 614). If Device App receives a YES acknowledgement, then the controller 502 proceeds to query another controller 510 or ambient sensor suite 222 for data. In various embodiments, instructions or steps for sensing, measurement, or ambient sensor data processing mode can be stored or updated in firmware within one or more local controller 510 or HVAC controller 502. In various embodiments, firmware instructions can include but are not limited to calling software libraries, initializing sensors, initializing device power, reading device IDs, pairing devices, determining sensor warmup time, acquiring sensor outputs, calibrating sensors, fault detection, collecting timestamps, sending data, and processing one or more recursive instructions.

Referring now to various embodiments of the application layer, including but not limited to applications stored in application server 504, data is posted from Device App using one or more APIs. In various embodiments, one or more scripts, one or more Control App 510 manages real-time acquisition, processing, and analysis of messages for subsequent storage to one or more database 506. In an embodiment, a posting script, posting API returns a response to the result of the post. The process checks whether data has been received successfully from one or more local controller 510, ambient sensor suite 222, or HVAC controller 502, stored on at least one computing stack. One or more scripts running, for example, on HVAC controller 502, can check the status of one or more stack, and manage a mechanism of message transmission or re-submission depending on the status of a communication network connection. In various embodiments, the message transfer protocol for one or more local controller 510 or HVAC controller 502 devices includes but is not limited to RESTful, compatible with HTTP for devices with limited resources such as battery capacity, low memory, or reduced processing capabilities. In various embodiments, the application layer protocol includes the constrain application protocol (CoAP). In various embodiments, ambient sensor suite 222, HVAC controller 502, router 520, or thermostat 510 may communicate via messages with at least one application (e.g., Control App 510), residing on application server 504, using protocol layer such as Message Queue Telemetry Transport (MQTT) to said connected devices with middleware and real-time applications. In various embodiments, said connected devices achieve real-time functions through the binding, bridge (e.g., ponte), or broker (e.g., MOTT broker) of one or more said protocols (e.g., CoAP, HTTP, MQTT, DTLS, UDP, XMPP, SMS, Web Socket, etc.). In various embodiments, one or more Device Management (DM) application residing on HVAC controller 510, one or more thermostat 510, or router 520, comprises the use of OAM DM or OMA Lightweight M2M (LWM2M) protocols or standards. DM functionalities include, but are not limited to, bootstrapping to automatically connect one or more connected devices to application server 504 using key management; device configuration to change parameters of the device and network settings; firmware updates; fault management for automatic error reporting; debugging; configuration; control applications; reporting, notification mechanism to alert for new data value; alarms and events. In various embodiments, controller 502, thermostat 510, router 520, or application server 504, one or more application 510, 526 employs the ETSI M2M standard interfaces (mla, dla and mld) and Service Capabilities Layers at one or more device, gateway, or network domains to achieve IoT interoperability. In various embodiments, one or more interworking proxy enables devices non-compliant with ETSI M2M by translating one or more specific protocol to another protocol (e.g., CoAP message to a specific HTTP POST message, etc.). In a various embodiment, real-time operation employs ETS M2M standard (e.g., NSCL, GSCL) whereby one or more connected devices of the present disclosure reports data to the GSCL enabling high-level applications at the network domain to retrieve data via NSCL. In various embodiments, application server 504, one or more applications 510, 526 interact with the GSCL via NSCL to discover registered applications (e.g., controllers 502, 510, etc.) to create a subscription to a particular resource. Application server 504 monitors one or more IP or port for incoming data. In one embodiment, when HVAC controller 502, one or more or local thermostat 510, or router 520 transmits new data, the GSCL automatically send an HTTP POST with a, but not limited to, XML encoded message containing a new reported data value to server 504, or one or more applications 510, 526.

An object of the present disclosure is a real-time monitoring of web application via application server 504. In various embodiments, one or more software module, for example Control App 510, including but not limited to Node.js modules, are adapted for environment occupant comfort sensing, diagnostics, analysis, and environmental control. In various embodiments, one or more Node.js modules employ an event loop, a thread pool, or combinations thereof. In one embodiment, the event loop is a single thread application. In another embodiment, input-output (I/O) functions are delegated to a thread pool under the control of an operating system (OS). In a preferred embodiment, the event loop continuously retrieves code from an event queue and executes it, excluding callbacks from previously stacked I/O tasks. Upon the completion of a previous I/O task, the event loop processes any callback. In various embodiments, the said software modules are fast and scalable 1/O bound applications containing simple low-level complex event loop and OS callbacks abstractions for built-in real time web monitoring. In various embodiments, processing time is reduced using one or more interworking proxy, including but not limited to, MQTT-binding and Web Sockets, ponte to bridge CoAP to MQTT, one or more MQTT broker, to bridge HTTP, CoAP, and MQTT messages, services, or the like.

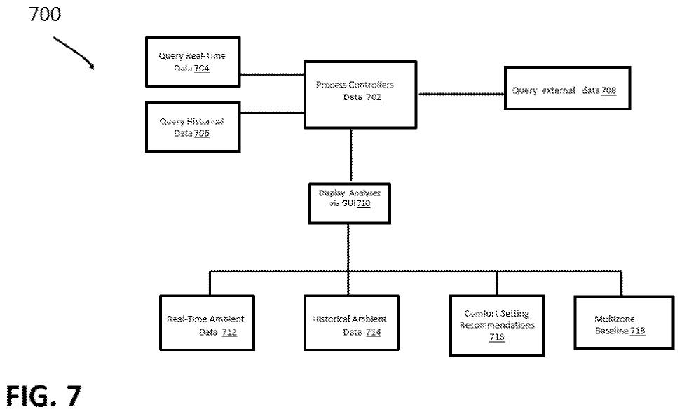

Referring now to FIG. 7, a functional block diagram of a routine 700 for occupant comfort zone environment analysis and control is shown. According to an embodiment of the present disclosure, routine 700 for occupant comfort zone environment analysis and control is initiated to process HVAC controller 502 and or local controller 510 data at the application server level 504 (step 702). The application server executes instructions to query real-time data of one or more said controllers from the application database 506 (step 704) and query historical controllers data from the application database 506 (step 706). The application server may also query regional or geographically proximal third-party weather data (step 708) to aggregate such data for one or more internal, external, or total load calculations. Application server 504 and one or more applications (e.g., Control App) residing on server processes the queried data from controller 502 and or local controller 510 to assemble an occupant comfort zone environment control analysis for one or more zone 512 and communicates and displays the analysis via user interface (step 710) executing on a client device 522, 524. The occupant comfort zone environment analysis and control provides one or more recommendations and or data visualizations for the user, including real-time ambient data (step 712), historical ambient data for the one or more zones (step 714), ambient data recommendations based on real-time and historical data (step 716), and ambient data comparisons between one or more zone and the multizone environment comparative data (step 718).

In summary, the AAD system and various embodiments of the present disclosure enable a dynamic control or reduction of the effective environmental and occupant load specifically within one or more zones of a multizone environment. The analyses of various environmental, occupant, or ambient parameters within various zones or rooms, as well as the presence of various individuals throughout the home overtime, enable the system to automatically distribute and control comfort settings specifically to individuals, distribute zones, a specific zone throughout the home and or provide energy conservation suggestions to the homeowner throughout the home at various points in the day, for example isolating certain rooms and reducing the load within the home for cooling/heating. The system uses automation and feedback control to achieve such benefits from the innovations of the present disclosure.

The above detailed description includes references to the accompanying drawings, which form a part of the detailed description. The drawings show, by way of illustration, specific embodiments in which the invention may be practiced. These embodiments are also referred to herein as “examples.” Such examples may include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

Method examples described herein may be machine or computer-implemented at least in part. Some examples may include a computer-readable medium or machine-readable medium encoded with instructions operable to configure an electronic device to perform methods as described in the above examples. An implementation of such methods may include code, such as microcode, assembly language code, a higher-level language code, or the like. Such code may include computer readable instructions for performing various methods. The code may form portions of computer program products. Further, in an example, the code may be tangibly stored on one or more volatile, non-transitory, or non-volatile tangible computer-readable media, such as during execution or at other times. Examples of these tangible computer-readable media may include, but are not limited to, hard disks, removable magnetic disks, removable optical disks, memory cards or sticks, random access memories (RAMs), read only memories (ROMs), and the like.

The above description is intended to be illustrative, and not restrictive. For example, the above-described examples (or one or more aspects thereof) may be used in combination with each other. Other embodiments may be used, such as by one of ordinary skill in the art upon reviewing the above description.

The Abstract is provided to comply with 37 C.F.R. § 1.72(b), to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment.

Thus, the following claims are hereby incorporated into the Detailed Description, with each claim standing on its own as a separate embodiment, and it is contemplated that such embodiments may be combined with each other in various combinations or permutations. The scope of the invention should be determined with reference to the appended claims, along with the full scope of equivalents to which such claims are entitled.

What is claimed is:

1. An adaptive air distribution control system comprising:
a plurality of indoor air quality sensors, the plurality of indoor air quality sensors being configured to continuously measure one or more indoor air quality parameters;
at least one duct actuator being configured to selectively regulate at least one airflow pathway;
a controller, the controller being operably engaged with the at least one duct actuator and communicably engaged with the plurality of indoor air quality sensors to receive a plurality of indoor air quality data inputs from the plurality of sensors, the controller comprising one or more processors, an input/output interface, and a non-transitory computer readable medium having stored thereon a set of instructions being programmable by a user and executable by at least one of the one or more processors, to cause the at least one of the one or more processors to perform one or more operations, comprising:
configuring one or more HVAC system components in response to the plurality of indoor air quality data inputs from the plurality of sensors;
performing a variable internal load calculation in response to the at least one duct actuator selectively regulating at least one airflow pathway; and
regulating an electrical current to the one or more HVAC system components in response to the variable internal load calculation; and
an application cloud server communicably engaged with the controller via a communications network, the application cloud server comprising an application software and an application database,
wherein the application cloud server is configured to query regional or geographically proximal third-party weather data to determine one or more internal, external or total load calculations.

2. The system of claim 1 further comprising at least one body-worn or body-carried occupant electronic device communicably engaged with the controller, the at least one body-worn or body-carried occupant electronic device being configured to measure one or more occupant-centric parameters.

3. The system of claim 2 wherein the one or more operations further comprise configuring one or more HVAC system components in response to one or more occupant-centric data inputs.

4. The system of claim 2 wherein the at least one body-worn or body-carried occupant electronic device comprises a wearable biometric sensor.

5. The system of claim 1 wherein the plurality of indoor air quality sensors are communicably engaged via a mesh network.

6. The system of claim 1 further comprising a smart phone operably engaged with the controller, the smart phone being configured to track a geolocation of an occupant-user.

7. The system of claim 1 further comprising one or more smart home sensors communicably engaged with the controller, the one or more smart home sensors being configured to measure one or more internal load parameters.

8. The system of claim 7 wherein the operations further comprise performing a variable internal load calculation in response to a plurality of data inputs from the one or more smart home sensors.

9. An adaptive air distribution system comprising:
an air handler comprising a housing, a variable speed blower, and at least one cooling coil;
a plurality of indoor air quality sensors, the plurality of indoor air quality sensors being configured to continuously measure one or more indoor air quality parameters;
at least one duct actuator being configured to selectively regulate at least one airflow pathway;
a controller, the controller being operably engaged with the air handler and the at least one duct actuator, and communicably engaged with the plurality of indoor air quality sensors to receive a plurality of indoor air quality data inputs from the plurality of sensors, the controller comprising one or more processors, an input/output interface, and a non-transitory computer readable medium having stored thereon a set of instructions being programmable by a user and executable by at least one of the one or more processors, to cause the at least one of the one or more processors to perform one or more operations, comprising:
configuring one or more HVAC system components in response to the plurality of indoor air quality data inputs from the plurality of sensors;
performing a variable internal load calculation in response to the at least one duct actuator selectively regulating at least one airflow pathway; and
regulating an electrical current to the variable speed blower and the at least one cooling coil in response to the variable internal load calculation; and
an application cloud server communicably engaged with the controller via a communications network, the application cloud server comprising an application software and an application database,
wherein the application cloud server is configured to query regional or geographically proximal third-party weather data to determine one or more internal, external or total load calculations.

10. The system of claim 9 further comprising an inverter operably engaged with the air handler.

11. The system of claim 9 further comprising at least one body-worn or body-carried occupant electronic device communicably engaged with the controller, the at least one body-worn or body-carried occupant electronic device being configured to measure one or more occupant-centric parameters.

12. The system of claim 9 further comprising a smart phone operably engaged with the controller, the smart phone being configured to track a geolocation of an occupant.

13. The system of claim 9 further comprising one or more smart home sensors communicably engaged with the controller, the one or more smart home sensors being configured to measure one or more internal load parameters.

14. The system of claim 11 wherein the at least one body-worn or body-carried occupant electronic device comprises a wearable biometric sensor.

15. The system of claim 9 further comprising one or more occupant proximity sensors communicably engaged with the controller.

16. An adaptive air distribution system comprising:
an air handler comprising a housing, a variable speed blower, and at least one cooling coil;
a plurality of indoor air quality sensors, the plurality of indoor air quality sensors being configured to continuously measure one or more indoor air quality parameters;
at least one body-worn or body-carried occupant electronic device being configured to measure one or more occupant comfort parameters;
at least one duct actuator being configured to selectively regulate at least one airflow pathway; and,
a controller, the controller being operably engaged with the air handler and the at least one duct actuator, the controller being communicably engaged with the plurality of indoor air quality sensors to receive a plurality of indoor air quality data inputs from the plurality of sensors, and being communicably engaged with at least one body-worn or body-carried occupant electronic device to receive a plurality of occupant comfort data inputs, the controller comprising one or more processors, an input/output interface, and a non-transitory computer readable medium having stored thereon a set of instructions being programmable by a user and executable by at least one of the one or more processors, to cause the at least one of the one or more processors to perform one or more operations, comprising:
configuring one or more HVAC system components in response to the plurality of indoor air quality data inputs and the plurality of occupant comfort data inputs;
performing a variable internal load calculation in response to the at least one duct actuator selectively regulating at least one airflow pathway; and
regulating an electrical current to the variable speed blower and the at least one cooling coil in response to the variable internal load calculation; and
an application cloud server communicably engaged with the controller via a communications network, the application cloud server comprising an application software and an application database,
wherein the application cloud server is configured to query regional or geographically proximal third-party weather data to determine one or more internal, external or total load calculations.

17. The system of claim 16 further comprising a smart phone communicably engaged with the application cloud server via the wireless network.

18. The system of claim 16 further comprising at least one third-party server communicably engaged with the application cloud server.

19. The system of claim 16 wherein the application software is configured to define one or more indoor environmental controls in response to real-time sensor data, historical sensor data, and external environmental data.
